(19) United States
(12) Patent Application Publication
(10) Pub. No.: US 2020/0315998 A1
(43) Pub. Date: Oct. 8, 2020

(54) COMPOSITION FOR IMPROVING INTESTINAL BARRIER FUNCTION

(71) Applicant: SUNTORY HOLDINGS LIMITED, Osaka-shi, Osaka (JP)

(72) Inventors: Ayuta Funaki, Soraku-gun (JP); Naoki Kasajima, Soraku-gun (JP); Hideyuki Arie, Soraku-gun (JP)

(73) Assignee: SUNTORY HOLDINGS LIMITED, Osaka-shi, Osaka (JP)

(21) Appl. No.: 16/957,577

(22) PCT Filed: Dec. 26, 2018
(86) PCT No.: PCT/JP2018/047840

§ 371 Date: Jun. 24, 2020

(30) Foreign Application Priority Data
Dec. 27, 2017 (JP) 2017-251995

(51) Int. Cl. A61K 31/192; A61K 9/00; A61K 31/05; A61K 31/7034

(52) U.S. Cl. CPC A61K 31/192; A61K 31/7034; A61K 31/05; A61K 9/0053

(57) ABSTRACT

An object of the present invention is to provide: a composition for improving intestinal barrier function capable of improving intestinal barrier function; and an agent for enhancing the intestinal barrier function improvement action of gallic acid. The present invention relates to a composition for improving intestinal barrier function, containing: gallic acid as an active ingredient.

### TECHNICAL FIELD

The present invention relates to a composition for improving intestinal barrier function. The present invention relates to an agent for enhancing an intestinal barrier function improvement action of gallic acid. The present invention also relates to a method for improving intestinal barrier function, and use of gallic acid for improving intestinal barrier function.

### BACKGROUND ART

In recent years, awareness with regard to intestinal health has increased, and a large number of intestine-related functional foods have been also sold. An intestinal function mainly includes a nutrient absorption function and a barrier function (intestinal barrier function) which prevents the intrusion (permeation) of toxic substances. Among these, it has become clear that the intestinal barrier function is deeply involved with chronic inflammation diseases increasing with aging.

Underneath intestinal epithelial cells, there exist a great number of immune cells such as macrophages, dendritic cells, T-cells, and B-cells. Typically, the intestinal epithelial cells are tightly joined to each other by a structure referred to as a tight junction, so that high molecular weight substances are strictly controlled so as not to pass through intercellular spaces. The intestinal epithelial cells contain transporters for removing hydrophobic foreign substances from the cells. The tight junction structure, the transporters and the like are responsible for the intestinal barrier function which prevents the intrusion of foreign substances. However, when the intestinal barrier is damaged by aging, neglect of health in life, stress and the like, leading to an increase in intestinal permeability, high molecular weight substances such as intestinal bacteria and their bacterial ingredients, which are present in the intestine, penetrate through intercellular spaces into the body and stimulate immune cells to release inflammatory cytokine, thereby inducing inflammation. As a result, various disease states caused by chronic inflammation such as inflammatory bowel disease and irritable bowel syndrome in the intestine, non-alcoholic fatty liver disease (NAFLD) in the liver, sarcopenia in the muscles, arteriosclerosis-induced symptoms in the blood vessel, cognitive decline, depression and anxiety in the brain, generalized diabetes, abnormal lipid metabolism, and autoimmune disease are considered to be induced. An undigested substance is assumed to intrude out of the intestine to induce allergies.

As described above, the reduction of the intestinal barrier function may cause various diseases. From such a viewpoint, searches for materials which can improve the intestinal barrier function have been attempted. An epithelial cell growth factor (EGF) has been known to promote the maturation of the intestinal epithelial cells, to enhance a barrier function. However, only a small amount of the EGF which is cytokine exists in the living body, so that it is not preferable to use the EGF as a material which improves the intestinal barrier function in respects of economic efficiency and safety. Non-Patent Literature 1 discloses that flavonoid such as quercetin promotes the formation of a tight junction or the like to prevent chronic inflammation. Patent Literature 1 discloses an absorption depressant which contains one or two or more selected from lindane, star anise, marnie, tea, black tea, or treated products thereof as active ingredient(s). Patent Literature 2 discloses that hexapeptide of a specific sequence and tryptophan have absorption suppressive activity of allergen. Patent Literature 3 discloses a supplement to be administered enterally to maintain or restore the intestinal barrier of the intestine, including a combination of glutamine, a substance having antioxidant activity, and a short-chain fatty acid.

### CITATION LIST

### Patent Literature

Patent Literature 1: JP 2002-193819 A
Patent Literature 2: JP 2002-257814 A
Patent Literature 3: JP 2004-513912 T

### Non-Patent Literature

Non-patent literature 1: Suzuki T. et al, The Journal of Nutritional Biochemistry. 2011 May, Vol. 22(5), p. 401-408

### SUMMARY OF INVENTION

### Technical Problem

An object of the present invention is to provide a composition for improving intestinal barrier function, the composition capable of improving intestinal barrier function. Another object of the present invention is to provide an agent for enhancing an intestinal barrier function improvement action of gallic acid.

### Solution to Problem

In order to solve the above problems, the present inventors intensively studied, and tried to solve the problems by adding inflammatory cytokine to an intestinal permeation model using human intestinal cell cultivated strain Caco-2, to produce a state where intestinal barrier function can be collapsed in humans, and finding a substance capable of ameliorating the state. As a result, gallic acid (3,4,5-trihydroxybenzoic acid) was found to have an intestinal barrier function improvement action. It was found that, when gallic acid and a phenolic compound other than gallic acid are used in combination, a synergistic effect thereof can provide an excellent intestinal barrier function improvement effect. It was a surprising and novel discovery to synergistically enhance the intestinal barrier function improvement effect when gallic acid and the phenolic compound are used in combination. The present inventors completed the present invention based on the findings.

That is, the present invention relates to the following composition for improving intestinal barrier function, and the like.

(1) A composition for improving intestinal barrier function, containing: gallic acid as an active ingredient.

(2) The composition for improving intestinal barrier function according to the above (1), further containing a phenolic compound.

(3) The composition for improving intestinal barrier function according to the above (2), wherein the phenolic compound is a polyphenol and/or a coumaric acid.

(4) The composition for improving intestinal barrier function according to the above (3), wherein the polyphenol is at least one compound selected from the group consisting of a flavan-3-ol polymer, flavanols, flavonols, flavanones, flavones, isoflavones, anthocyanidins, flavanonols, stilbenoids, chalcones, and a hydrolyzable tannin.

(5) The composition for improving intestinal barrier function according to any one of the above (2) to (4), wherein the phenclic compound is at least one compound selected from the group consisting of procyanidin B1, procyanidin B2, procyanidin B3, catechin, epicatechin, gallocatechin, epigallocatechin, catechin gallate, epicatechin gallate, epigallocatechin gallate, theaflavin, taxifolin, daidzein, genistein, apigenin, luteolin, naringenin, naringenin chalcone, kaempferol, rutin, quercetin-3-O-glucopyranoside, quercetin, myricetin, piceatannol, petunidin, trans-piceid, corilagin, stenophyllanin A, stenophyllanin B, casuarinin, geraniin, tellimagrandin I, pedunculagin, praecoxin A, eugeniflorin D2,1,4,6-tri-O-galloyl-β-D-glucose, 1,2,3,6-tetra-O-galloyl-β-D-glucose, 2,3,4,6-tetra-O-galloyl-β-D-glucose, 1,2,4,6-tetra-O-galloyl-β-D-glucose, 1,2,3,4,6-penta-O-galloyl-β-D-glucose, β-glucogallin, 2-coumaric acid, and 3-coumaric acid.

(6) The composition for improving intestinal barrier function according to any one of the above (1) to (5), wherein the composition for improving intestinal barrier function is an oral composition.

(7) The composition for improving intestinal barrier function according to the above (6), wherein the oral composition is a food or beverage, a pharmaceutical product, or a quasi-pharmaceutical product.

(8) The composition for improving intestinal barrier function according to any one of the above (1) to (7), wherein the composition is used for intestinal regulation.

(9) The composition for improving intestinal barrier function according to any one of the above (1) to (8), wherein the composition is labeled as having an intestinal regulation action.

(10) An agent for enhancing an intestinal barrier function improvement action of gallic acid, the agent containing: a phenolic compound as an active ingredient.

(11) A method for improving intestinal barrier function, the method including: administering gallic acid to a subject.

(12) Use of gallic acid for improving intestinal barrier function.

(13) A method for enhancing an intestinal barrier function improvement action of gallic acid, the method including: administering a combination of gallic acid with a phenolic compound to a subject.

(14) Use of a phenolic compound for enhancing an intestinal barrier function improvement action of gallic acid.

### Advantageous Effects of Invention

The use of a composition for improving intestinal barrier function according to the present invention makes it possible to improve intestinal barrier function. The use of an agent for enhancing an intestinal barrier function improvement action of the present invention makes it possible to significantly enhance an intestinal barrier function improvement action of gallic acid. The present invention improves the intestinal barrier function, which makes it possible to contribute also to prevention or treatment of conditions or diseases related to intestinal barrier dysfunction such as chronic inflammatory diseases and allergy diseases.

### DESCRIPTION OF EMBODIMENTS

A composition for improving intestinal barrier function according to the present invention contains gallic acid as an active ingredient.

Gallic acid has an intestinal barrier function improvement action. Gallic acid is an ingredient contained in a plant such as grape or tea plant, and has few side effects and high safety even if gallic acid is ingested for a long period of time.

Gallic acid is not limited by the derivation or the producing method. For example, plant-derived gallic acid extracted from a plant may be used, and gallic acid obtained by a synthetic method may be used.

It is preferable that the composition for improving intestinal barrier function according to the present invention further contains a phenolic compound. One of the phenolic compounds may be used, and two or more thereof may be used.

In the present invention, the phenolic compound refers to a compound having a phenolic hydroxyl group other than gallic acid, or its glycoside. Gallic acid is not included in the phenolic compound in the present invention.

The glycoside refers to a compound which is formed by the glycoside-bonding of the hydroxyl group of a sugar to a non-sugar compound. The sugar in the glycoside is not limited. It may be a monosaccharide, a di- or higher saccharide, or a mixture of sugars. The type of the sugar is also not limited, and can be exemplified by aldoses such as glucose, mannose, galactose, fucose, rhamnose, arabinose and xylose; ketoses such as fructose; uronic acids such as glucuronic acid, galacturonic acid and mannuronic acid; as well as apiose and rutinose. The sugar in the glycoside may be a D-sugar or an L-sugar.

The combination of gallic acid with the phenolic compound makes it possible to enhance the intestinal barrier function improvement action of gallic acid. Therefore, the use of the combination can provide an excellent intestinal barrier function improvement effect. The intestinal barrier function improvement effect obtained by the combination of gallic acid with the phenolic compound is a synergistic effect which is vastly superior to an additive effect predicted from an effect obtained by independently using each ingredient.

In one aspect of the present invention, the composition for improving intestinal barrier function is preferably a composition for improving intestinal barrier function which contains gallic acid and a phenolic compound as active ingredients.

Examples of the phenolic compound used in the present invention include a polyphenol and a coumaric acid. The polyphenol refers to a compound having two or more phenolic hydroxyl groups in its molecule, or its glycoside. Examples of the coumaric acid include p-coumaric acid, 2-coumaric acid, 3-coumaric acid, and their glycosides.

The polyphenol is preferably a flavan-3-ol polymer, flavanols, flavonols, flavanones, flavones, isoflavones, anthocyanidins, flavanonols, stilbenoids, chalcones, a hydrolyzable tannin and the like. The use of the polyphenol makes it possible to enhance the intestinal barrier function improvement action of gallic acid.

The flavan-3-ol polymer contains flavan-3-ol as a structural unit, and is a dimer or higher polymer with 4-6 or 4-8 interflavan bonds formed by condensation or polymerization. The flavan-3-ol polymer is a compound referred to as condensed tannin. The flavan-3-ol polymer may be a mixture of two or more polymers having different degrees of polymerization. In one aspect, the flavan-3-ol polymer may have a galloyl group. The degree of polymerization of the flavan-3-ol polymer is not limited. For example, a flavan-3-ol polymer having a degree of polymerization of 2 to 30 (2 to 30-mers) can be used. In one aspect, the flavan-3-ol polymer is preferably a flavan-3-ol dimer. Examples of the flavan-3-ol dimer include procyanidin B1, procyanidin B2, and procyanidin B3.

Examples of the compounds of the flavanols include flavan-3-ols such as catechin, epicatechin, gallocatechin, epigallocatechin, catechin gallate, epicatechin gallate, gallocatechin gallate, and epigallocatechin gallate; and theaflavin. In terms of an excellent action of enhancing the intestinal barrier function improvement action when the flavanols are used in combination with gallic acid, the flavanols are preferably catechin, epicatechin, gallocatechin, epigallocatechin, catechin gallate, epicatechin gallate, epigallocatechin gallate, theaflavin and the like. In one aspect, in terms of providing a superior intestinal barrier function improvement effect when the flavanols are used in combination with gallic acid, the flavanols are more preferably catechin gallate, epicatechin gallate, epigallocatechin gallate and the like.

The flavan-3-ol polymer is not included in the flavanols in the present invention.

Examples of the compounds of the flavonols include quercetin, myricetin, kaempferol, and their glycosides (quercetin-3-O-glucopyranoside, rutin and the like). In terms of an excellent action of enhancing the intestinal barrier function improvement action, the flavonols are preferably quercetin, kaempferol, and their glycosides.

Examples of the compounds of the flavanones include naringenin and its glycoside.

Examples of the compounds of the flavones include apigenin, luteolin, and their glycosides.

Examples of the compounds of the isoflavones include daidzein, genistein, and their glycosides.

Examples of the compounds of the anthocyanidins include petunidin and its glycoside.

Examples of the compounds of the flavanonols include taxifolin and its glycoside.

Examples of the compounds of the stilbenoids include piceatannol and its glycosides (trans-piceid, cis-piceid and the like).

Examples of the compounds of the chalcones include naringenin chalcone (4,2′,4′,6′-tetra-hydroxy chalcone) and its glycoside.

Examples of the hydrolyzable tannin include gallotannin and ellagitannin. Examples of the gallotannin include β-glucogallin, 1,4,6-tri-O-galloyl-β-D-glucose, 1,2,4,6-tetra-O-galloyl-β-D-glucose, 1,2,3,6-tetra-O-galloyl-β-D-glucose, 2,3,4,6-tetra-O-galloyl-β-D-glucose, and 1,2,3,4,6-penta-O-galloyl-β-D-glucose. Examples of the ellagitannin include corilagin, tellimagrandin I, pedunculagin, praecoxin A, geraniin, stenophyllanin A, stenophyllanin B, casuarinin, and eugeniflorin D2.

In one aspect of the present invention, since a higher intestinal barrier function improvement effect can be obtained, the phenolic compound is preferably a flavan-3-ol polymer, flavanols, and ellagitannin. The phenolic compound is preferably a flavan-3-ol polymer (preferably, procyanidin B1, procyanidin B2, procyanidin B3 and the like), catechin, epicatechin, gallocatechin, epigallocatechin, catechin gallate, epicatechin gallate, epigallocatechin gallate, theaflavin, taxifolin, daidzein, genistein, apigenin, luteolin, naringenin, naringenin chalcone, kaempferol, rutin, quercetin-3-O-glucopyranoside, quercetin, myricetin, piceatannol, petunidin, trans-piceid, corilagin, stenophyllanin A, stenophyllanin B, casuarinin, geraniin, tellimagrandin I, pedunculagin, praecoxin A, eugeniflorin D2,1,4,6-tri-O-galloyl-β-D-glucose, 1,2,3,6-tetra-O-galloyl-β-D-glucose, 2,3,4,6-tetra-O-galloyl-O-D-glucose, 1,2,4,6-tetra-O-galloyl-β-D-glucose, 1,2,3,4,6-penta-O-galloyl-β-D-glucose, β-glucogallin, 2-coumaric acid, and 3-coumaric acid, more preferably procyanidin B1, procyanidin B2, procyanidin B3, catechin, epicatechin, gallocatechin, epigallocatechin, catechin gallate, epicatechin gallate, epigallocatechin gallate, theaflavin, taxifolin, 2-coumaric acid, daidzein, genistein, apigenin, luteolin, naringenin, naringenin chalcone, kaempferol, rutin, quercetin-3-O-glucopyranoside, quercetin, petunidin, trans-piceid, corilagin, stenophyllanin A, geraniin, tellimagrandin I, pedunculagin, 1,2,3,6-tetra-O-galloyl-β-D-glucose, 2,3,4,6-tetra-O-galloyl-β-D-glucose, 1,4,6-tri-O-galloyl-β-D-glucose, and β-glucogallin. When the phenolic compound is used in combination with gallic acid, the intestinal barrier function improvement action of gallic acid can be further enhanced.

The phenolic compound is not limited by the derivation or the producing method. For example, a plant-derived phenolic compound extracted from a plant may be used, and a phenolic compound obtained by a synthetic method may be used. Preferably, a plant-derived phenolic compound is used. For example, the flavan-3-ol polymer can be obtained from plants such as grape (preferably grape seed), cocoa, apple, pine, aronia, and lychee. The flavanols can be obtained from green tea leaves, tea leaves and the like. The hydrolyzable tannin can be obtained, for example, in a method of extracting a raw material containing a hydrolyzable tannin with water or hydrous ethanol, filtering the extract, removing the alcohol, and thereafter performing column purification. A plant containing a hydrolyzable tannin can be used as the raw material of the hydrolyzable tannin. Examples of the plant containing a hydrolyzable tannin include fagaceae, lythraceae, myrtaceae, and rosaceae plants. These plants are rich in hydrolyzable tannins. The myrtaceae plant is preferably a plant such as a syzygium, eucalyptus, or kunzea plant. For example, an extract of eucalyptus leaves is rich in hydrolyzable tannins such as tellimagrandin I and gallotannin.

Commercially available phenolic compounds can also be used.

In the present invention, the “intestinal barrier function” means the function of protecting the intrusion (permeation) of foreign materials (for example, toxins such as endotoxin, inflammatory substances, and undigested products) into the body from the outside of intestinal epithelial cells (the inside of the intestine). The large intestine and the small intestine are contained in the intestine. A state where the intrusion of the foreign materials into the body from the outside of the intestinal epithelial cells is promoted as compared with the normal state is referred to as a state where the permeability of the foreign materials in the intestinal epithelial cells increases (rises). The “intestinal barrier function improvement” means both suppression of the increase in the permeability of the foreign materials in the intestinal epithelial cells and decrease in the permeability of the foreign materials in the intestinal epithelial cells. In the present invention, the “intestinal barrier function improvement” is used to mean to include suppression of the decrease in the intestinal barrier function and enhancement of the decreased intestinal barrier function.

For example, the intestinal barrier function is improved by normalizing or strengthening a tight junction bonding the intestinal epithelial cells together. In one aspect, the composition for improving intestinal barrier function according to the present invention may be used in order to improve the intestinal barrier function by normalizing or strengthening the tight junction in the intestinal epithelial cells.

The intestinal barrier function improvement effect is indicated by, for example, increase in the electrical resistance value (transepithelial electric resistance: TEER) of the intestinal epithelial cells or suppression of the decrease in the TEER. A substance increasing the TEER or suppressing the decrease thereof has the action of normalizing or strengthening the tight junction in the intestinal epithelial cells. The intestinal barrier function improvement effect is also indicated by decrease in the amount of a substance permeating from the intestinal side of the intestinal epithelial cells to the inside of the body. The person skilled in the art can select a specific method of evaluating the intestinal barrier function improvement effect depending on the purpose. For exarple, as shown in Examples to be described later, a method for measuring TEER using an intestinal permeation model using human intestinal epithelial cells (Caco-2 cells) can be used. Specifically, inflammatory cytokine (TNFα, IL-1β, IFNγ and the like) is added into Caco-2 monolayer cultured cells to produce a state where the intestinal barrier function can be collapsed in human. If the addition of a test substance suppresses the decrease in the TEER as compared with the case where the substance is not added, the test substance can be evaluated to have the intestinal barrier function improvement effect.

As shown in Examples, gallic acid suppresses the decrease in the TEER due to the addition of inflammatory cytokine in an intestinal permeation model using Caco-2 cells to have an intestinal barrier function action. Gallic acid can normalize or strengthen the tight junction in the intestinal epithelial cells to improve the intestinal barrier function. As shown in Examples, the combination of gallic acid with the phenolic compound can provide a superior intestinal barrier function improvement effect.

The composition for improving intestinal barrier function according to the present invention contains gallic acid as the active ingredient to exhibit an intestinal barrier function improvement effect. When the composition for improving intestinal barrier function contains the phenolic compound, the intestinal barrier function improvement effect is enhanced, whereby a superior intestinal barrier function improvement effect can be provided.

For this reason, the composition for improving intestinal barrier function according to the present invention is useful for preventing or ameliorating conditions or diseases against which improvement of the intestinal barrier function is effective, for example, conditions or diseases related to the intestinal barrier dysfunction. The intestinal barrier dysfunction includes deterioration of the intestinal barrier function. Examples of the conditions or diseases related to the intestinal barrier dysfunction include conditions or diseases caused by the intestinal barrier dysfunction, or conditions or diseases involving the intestinal barrier dysfunction. Examples of the conditions or diseases related to the intestinal barrier dysfunction include inflammatory bowel diseases, irritable bowel syndromes, systemic autoimmune diseases (rheumatoid arthritis, erythematosus and the like), allergies (food allergy, pollinosis and the like), and metabolic syndromes (obesity, type I or type II diabetes mellitus, hypertension, hyperlipidemia, non-alcoholic fatty liver diseases (NAFLD), arteriosclerosis and the like) (for example, Camilleri et al., Am, J Physiol Gastrointest Liver Physiol. 303: G775-G785, 2012; Mu et al., Front. Immunol., Vol. 8, Article 598, 2017; Bischoff et, al., BMC Gastroenterology 2014 14:189).

More specific examples of symptoms of the conditions or diseases related to the intestinal barrier dysfunction include symptoms such as diarrhea, constipation, and discomfort (bloating, borborygmi, abdominal pain and the like) of the gut (abdominal part). The composition for improving intestinal barrier function according to the present invention has the action of improving the intestinal condition by improving the intestinal barrier function. Therefore, the composition for improving intestinal barrier function according to the present invention can control the intestinal function by the intestinal barrier function improvement, and is useful for preventing or ameliorating the symptom of the intestine as described above. In one aspect, the composition for improving intestinal barrier function according to the present invention may be used for intestinal regulation (in order to prevent or ameliorate, for example, diarrhea, constipation, and abdominal discomfort). The composition for improving intestinal barrier function according to the present invention is useful for intestinal regulation by improving the intestinal barrier function. The intestinal barrier dysfunction is also associated with metabolic syndromes and the like (for example, the Bischoff et al., BMC Gastroenterology 2014 14:189 as described above). It is effective also in prevention or amelioration of the metabolic syndromes to improve the intestinal barrier function. Examples of the symptoms of the metabolic syndromes include abnormal glucose metabolism, abnormal lipid metabolism, increase in body fat, increase in visceral fat, increase in abdominal circumference fat, and higher blood pressure. Therefore, the composition for improving intestinal barrier function according to the present invention can improve the intestinal barrier function to contribute to improvement of glucose metabolism, improvement of lipid metabolism, decrease or suppression of increase in fat such as body fat, visceral fat, or abdominal circumference fat, amelioration of higher blood pressure and the like.

Herein, the “prevention of conditions or diseases” refers to enhancement of the resistance of a subject to the conditions or diseases, or delay or prevention of the onset of the conditions or diseases. Herein, the “amelioration of conditions or diseases” refers to recovery of a subject from the conditions or diseases, alleviation of the symptoms of the conditions or diseases, or delay or prevention of the progress of the conditions or diseases.

The composition of the present invention is applicable for both therapeutic use (medical use) and non-therapeutic use (non-medical use).

The composition for improving intestinal barrier function according to the present invention can be provided, for example, as a food or beverage, a pharmaceutical product, a quasi-pharmaceutical product, feed or the like, but it is not limited thereto. The composition for improving intestinal barrier function according to the present invention may be a food or beverage, a pharmaceutical product, a quasi-pharmaceutical product, or feed by itself, and may be a formulation or material such as an additive used therefor. The composition for improving intestinal barrier function according to the present invention may be provided as an agent as one example, but it is not limited thereto. The agent may be provided as a composition by itself or as a composition containing the agent.

In one aspect, the composition for improving intestinal barrier function according to the present invention is preferably an oral composition. The present invention can provide an oral composition having an excellent intestinal barrier function improvement action. Examples of the oral composition include a food or beverage, a pharmaceutical product, and a quasi-pharmaceutical product, and the oral composition is preferably a food or beverage.

The composition for improving intestinal barrier function according to the present invention may contain one or two or more ingredients (other ingredients) other than the above-described gallic acid and the phenolic compound optionally blended as long as the effect of the present invention is not impaired.

In one aspect, for example, ingredients such as lactic acid bacteria, bifidobacteria, dietary fibers, and polysaccharides may be contained as the other ingredients.

It is preferable that the lactic acid bacteria and the bifidobacteria can be orally ingested.

The dietary fiber may be any of a water-insoluble dietary fiber and a water-soluble dietary fiber. Examples of the water-insoluble dietary fiber include cellulose, lignin, hemicellulose, wheat bran, an apple fiber, a sweet potato fiber, and chitin. The water-soluble dietary fiber is roughly divided into a high viscous dietary fiber and a low viscous dietary fiber, and examples of the high viscous dietary fiber include pectin, konjac mannan, alginic acid, sodium alginate, guar gum, and agar. Among the dietary fibers generally known in Japan, the low viscous water-soluble dietary fiber is referred to as a dietary fiber material containing 50% by weight or more of a dietary fiber and dissolved in ordinary temperature water to provide a low viscous solution, i.e., an about 5% by weight aqueous solution having a viscosity of 20 mPa-s or less. Examples of the low viscous water-soluble dietary fiber include hardly digestible dextrin, polydextrose, partially hydrolyzed guar gum, and Litesse (polydextrose). Other examples of the low viscous water-soluble dietary fiber include dietary fiber materials satisfying low viscous and water-soluble properties. One of the dietary fibers may be used, and two or more thereof may be used.

Examples of the polysaccharides include oligosaccharides such as galactooligosaccharides, xylooligosaccharides, mannooligosaccharides, agarooligosaccharides, fructooligosaccharides, isomaltooligosaccharides, and raffinose. One of the oligosaccharides may be used, and two or more thereof may be used.

The composition for improving intestinal barrier function according to the present invention may contain optional additives and optional ingredients other than the above. The additives and the ingredients may be selected depending on the form of the composition for improving intestinal barrier function, and the like. The additives and the ingredients generally usable for a food or beverage, a pharmaceutical product, a quasi-pharmaceutical product, feed or the like can be used. Examples thereof include various additives which are acceptable in a food or beverage or pharmaceutically acceptable as an oral administration agent, such as an excipient, a lubricant, a stabilizer, a dispereant, a binder, a diluent, a flavour, a sweetener, a flavoring agent, and a colorant. For example, when the composition for improving intestinal barrier function according to the present invention is used as the oral composition, the oral composition can appropriately contain ingredients capable of being orally ingested other than the above as long as the effect of the present invention is not impaired. Such ingredients include vitamin, a vitamin-like substance, protein, amino acid, fat and oil, organic acid, a carbohydrate, a plant-derived raw material, an animal-derived raw material, a microorganism, an additive for food or beverage, and an additive for pharmaceutical product.

In addition to the above, ingredients such as materials used for a food or beverage, a pharmaceutical product, a quasi-pharmaceutical product, feed or the like can be appropriately blended depending on the use.

The form of the composition for improving intestinal barrier function according to the present invention is not limited as long as the effect of the present invention is obtained. Examples of the form include tablets, pills, granules, fine granules, chews, capsules (including soft and hard capsules), liquids, chewable tablets, and beverages. The form may be other food form. These dosage forms may be prepared by using conventional methods commonly known in the art.

In one aspect, when the composition for improving intestinal barrier function according to the present invention is used as a food or beverage, ingredients (for example, a material for food or beverage, and an additive used as necessary) usable for the food or beverage can be blended with gallic acid and the phenolic compound optionally blended to provide various foods or beverages (compositions for food or beverage). The food or beverage is not limited. Examples thereof include general foods and beverages, health foods, foods with function claims, foods for specified health uses, foods for the sick, food additives, and raw materials thereof. The form of the food or beverage is not also limited, and examples thereof include various formulation forms such as solid oral formulations (such as tablets, coating tablets, fine granules, granules, powders, pills, capsules (including soft and hard capsules), dry syrup agents, and chewable tablets); and liquid oral formulations (such as internal liquid formulations and syrups). In one aspect of the present invention, the food or beverage may contain one or two or more of the lactic acid bacteria, the bifidobacteria, the dietary fibers, and the polysaccharides.

When the composition for improving intestinal barrier function according to the present invention is used as the pharmaceutical product or the quasi-pharmaceutical product, an additive such as a pharmaceutically acceptable excipient can be blended with gallic acid and the phenolic compound optionally blended, to provide the pharmaceutical products (pharmaceutical compositions) or quasi-pharmaceutical products (quasi-pharmaceutical product compositions) of various dosage forms. The form of administration of the pharmaceutical product or quasi-pharmaceutical product is preferably oral administration. The dosage form of the pharmaceutical product or quasi-pharmaceutical product may be a dosage form suitable to the form of administration. Examples of the dosage form of the oral pharmaceutical product or quasi-pharmaceutical product include solid oral formulations such as tablets, coating tablets, fine granules, granules, powders, pills, capsules (including soft and hard capsules), dry syrup agents, and chewable tablets; and liquid oral formulations such as internal liquid formulations and syrups.

The tablets, the pills, and the granules may be in dosage forms conventionally coated as necessary such as sugar-coated tablets, gelatin-coated preparations, enteric-coated preparations, and film-coated agents. The tablets may be in the form of double or multiple layer tablets.

When the composition for improving intestinal barrier function according to the present invention is used as a food or beverage, a pharmaceutical product, a quasi-pharmaceutical product, feed or the like, the producing method is not limited, and the composition for improving intestinal barrier function can be produced by a general method using gallic acid and the phenolic compound optionally blended. The present invention also includes use of gallic acid for the manufacture of the composition for improving intestinal barrier function. The present invention also includes use of gallic acid and the phenolic compound other than gallic acid for the manufacture of the composition for improving intestinal barrier function. In the present invention, the composition for improving intestinal barrier function can also be prepared using a plant extract containing gallic acid and a phenolic compound. The plant extract containing gallic acid and a phenolic compound is not limited, and for example, a grape seed extract, a green tea extract, an oolong tea extract, a black tea extract, an eucalyptus extract, a guava extract, a sweet tea extract, or a rose hip extract can be used.

The composition for improving intestinal barrier function according to the present invention may be labeled with one or more of the following information on a package, container, or package insert: usage, types of active ingredients, the above-described effects, and instructions for use (e.g., ingestion method or administration method). The composition for improving intestinal barrier function according to the present invention may be labeled as having an intestinal barrier function improvement action or an action based on the intestinal barrier function improvement action. The composition for improving intestinal barrier function may be labeled as having an intestinal regulation action, for example.

The intestinal regulation action may be an intestinal regulation action based on improvement of intestinal barrier function, and is not limited. Examples of the labeling as having an intestinal regulation action include “for one tending to be constipated or have diarrhea”, “for one worrying about gut function”, “for one readily feeling discomfort of gut”, “improvement of bowel movement”, “improvement of stool state”, “amelioration of defecation frequency”, “amelioration of defecation output”, “gut feels better”, “controlling gut function”, “controlling intestinal function”, “amelioration of discomfort in gut”, “reduction of generation of gas”, “reduction of abdominal bloatedness”, and “amerilation of borborygmi”. The composition for improving intestinal barrier function according to the present invention may include one or two or more of such labelings.

The content of gallic acid in the composition for improving intestinal barrier function according to the present invention can be appropriately set depending on the form of the composition and the like. In one aspect, when the composition for improving intestinal barrier function is used as the oral compositions such as a food or beverage, a pharmaceutical product, and a quasi-pharmaceutical product, the content of gallic acid is preferably 0.0001% by weight or more, more preferably 0.001% by weight or more in the composition. The content is preferably 10% by weight or less, more preferably 1% by weight or less in the composition. In one aspect, the content of gallic acid is preferably 0.0001 to 10% by weight, more preferably 0.001 to 1% by weight in the composition for improving intestinal barrier function.

The content of gallic acid can be measured according to known methods, and for example, an HPLC method can be used.

When the composition for improving intestinal barrier function according to the present invention contains the phenolic compound, the molar ratio of the contents of gallic acid and the phenolic compound (gallic acid/phenolic compound) is preferably 0.1 to 10, more preferably 0.3 to 5. If gallic acid and the phenolic compound are used at the above ratio, the intestinal barrier function improvement effect can be further improved.

The composition for improving intestinal barrier function according to the present invention can be ingested or administered by a suitable method depending on the form. It is preferable that the composition for improving intestinal barrier function according to the present invention is orally administered or orally ingested.

The amount of the composition for improving intestinal barrier function according to the present invention ingested (which may also be referred to as the amount administered) is not limited, and may be an amount such that the intestinal barrier function improvement effect is obtained. The amount ingested may be appropriately set according to the form of administration or administration method, for example. As one aspect, for the amount of the composition for improving intestinal barrier function ingested when the composition is orally administered to or allowed to be ingested by a human (adult) subject, the amount of gallic acid ingested is preferably 0.01 to 500 mg, more preferably 0.1 to 300 mg, still more preferably 1 to 100 mg per day. It is preferable that the amount is orally administered or allowed to be ingested, for example, once daily or in a divided manner two to three times daily. When the composition for improving intestinal barrier function is allowed to be ingested by the human (adult) subject for the purpose of obtaining the intestinal barrier function improvement effect, the composition for improving intestinal barrier function is preferably orally ingested by or administered to the subject so that the amount of gallic acid ingested is within the above range.

In one aspect, the composition for improving intestinal barrier function according to the present invention preferably contains gallic acid in an amount such that the desired effect of the present invention is obtained, that is, an effective amount in consideration of the form of administration, administration method and the like thereof. As one aspect, for example, when the composition for improving intestinal barrier function is an oral composition, the content of gallic acid in the amount of the composition ingested per adult per day is preferably 0.01 to 500 mg, more preferably 0.1 to 300 mg, still more preferably 1 to 100 mg. In one aspect of the present invention, the composition for improving intestinal barrier function preferably contains a phenolic compound in addition to the above amount of gallic acid.

A subject (hereinafter, merely referred to as an “administration subject”) to whom the composition for improving intestinal barrier function according to the present invention is administered or by whom the composition is allowed to be ingested is preferably human or non-human animal, more preferably mammal (human or non-human mammal), still more preferably human. The administration subject in the present invention is preferably a subject requiring or desiring the intestinal barrier function improvement. Suitable examples of the subject include a subject having a reduced intestinal barrier function and a subject desiring the prevention or amelioration of conditions or diseases related to intestinal barrier dysfunction.

The present invention also includes the following method for improving intestinal barrier function: a method for improving intestinal barrier function, the method including administering gallic acid to a subject.

In the method for improving intestinal barrier function, it is preferable to further administer a phenolic compound. By administering a combination of gallic acid with the phenolic compound, an excellent intestinal barrier function improvement effect is obtained. A preferable aspect of the phenolic compound is the same as that in the case of the composition for improving intestinal barrier function.

When gallic acid and the phenolic compound are administered, these may be separately administered, or may be simultaneously administered. Preferably, gallic acid and the phenolic compound are simultaneously administered. Gallic acid and the phenolic compound optionally used may be administered as it is, and a composition containing gallic acid and the like may be administered. For example, the above-described composition for improving intestinal barrier function according to the present invention can be administered.

The present invention also includes the following use: use of gallic acid for improving intestinal barrier function.

In the above use, it is preferable to use gallic acid and a phenolic compound.

In the above method for improving intestinal barrier function and the use, gallic acid, the phenolic compound optionally used, the subject (administration subject), the administration method, the amount administered, preferable aspects thereof and the like are the same as those in the case of the above-described composition for improving intestinal barrier function.

For example, the amount of gallic acid administered may be an amount at which the intestinal barrier function improvement effect is obtained, i.e., an effective amount, and is not limited. For example, the above-described amount of gallic acid can be administered. When the phenolic compound is used, the molar ratio of gallic acid and the phenolic compound (gallic acid/phenolic compound) is preferably 0.1 to 10, more preferably 0.3 to 5. When the ratio of gallic acid and the phenolic compound is within the above range, the intestinal barrier function improvement effect can be further improved.

As described above, the intestinal barrier function improvement action of gallic acid can be enhanced by using a combination of gallic acid with the phenolic compound. The above phenolic compound may be used in order to enhance the intestinal barrier function improvement action of gallic acid.

Another aspect of the present invention is an agent for enhancing an intestinal barrier function improvement action of gallic acid, the agent containing a phenolic compound as an active ingredient; and use of a phenolic compound for enhancing the intestinal barrier function improvement action of gallic acid.

The present invention also includes a method for enhancing an intestinal barrier function improvement action of gallic acid, the method including administering a combination of gallic acid with a phenolic compound to a subject.

In the agent for enhancing an intestinal barrier function improvement action, the method for enhancing the intestinal barrier function improvement action, and the like, the phenolic compound, its preferable aspect and the like are the same as those in the case of the above-described composition for improving intestinal barrier function. A preferable ratio of the amounts of gallic acid and the phenolic compound used is also the same as that in the case of the above-described composition for improving intestinal barrier function. The molar ratio of gallic acid and the phenolic compound (gallic acid/phenolic compound) is preferably 0.1 to 10, more preferably 0.3 to 5. When the ratio of gallic acid and the phenolic compound is within the above range, the intestinal barrier function improvement action can be further enhanced.

The method and the use may be therapeutic or non-therapeutic. The “non-therapeutic” is a concept which does not include medical activities, i.e., a concept which does not include surgery, therapy or diagnosis.

### EXAMPLES

The following provides Examples which more specifically describe the present invention. The present invention is not limited to these Examples.

### <Method for Evaluating Intestinal Barrier Function Improvement Action>

In Examples, the intestinal barrier function improvement action of a test compound (hereinafter, referred to as a sample) was evaluated by the following method using Caco-2 cells.

### Evaluation of Ingredient Improving Intestinal Barrier Function Using Caco-2 Cells

Caco-2 cells were cultured for three weeks at 37° C. in Transwell (manufactured by Millicell Corporation) using DMEM (Dulbecco's modified Eagle's medium). A medium was removed from a plate of cultured Caco-2 cells, and the well was washed 3 times with serum-free DMEM. The well was filled with the medium. Then, the transepithelial electrical resistance (TEER) of Caco-2 monolayer cells was measured by Millicell-ERS (manufactured by Millipore Corporation). Cells (TEER≥1000 Ω·cm²) determined that a sufficient tight junction was formed were selected, and used for the next screening. Then, to test solutions (media) on both apical and basolateral membrane sides, a sample, TNFα (40 ng/mL), IL-1β (20 ng/mL), and IFNγ (10 ng/mL) were added, followed by cultivating for 48 hours. The sample was dissolved in dimethyl sulfoxide (DMSO), and the solution was then added to the test solutions. In this case, a well to which inflammatory cytokine (TNFα, IL-1β, and IFNγ) and the sample were not added was provided as normal. A well to which inflammatory cytokine was added and the sample was not added was provided as control. After the cultivation, TEER was measured again, to evaluate whether the sample suppressed the reduction (decrease) of TEER due to the inflammatory cytokine.

The TEER reduction suppression ratio (%) due to the sample was determined according to the following formula from the TEER values of the well to which the sample was added, normal, and control.

(Calculating formula of TEER reduction suppression ratio) TEER reduction suppression ratio (%)=100×((TEER of well to which sample is added)−(TEER of control))/((TEER of normal)−(TEER of control))

In this evaluation system, as the TEER reduction suppression ratio (%) is higher, an intestinal barrier function improvement effect is higher.

### Example 1

An intestinal barrier function improvement action in Caco-2 cells was evaluated according to the above evaluation method using gallic acid as a sample. Gallic acid (Nacalai Tesque, Inc.) was added so that a concentration in the test solution was set to 100 μM (μmol/L).

As reference, 100 μM of quercetin (Funakoshi Co., Ltd.) was added into the test solution instead of gallic acid, to similarly evaluate the intestinal barrier function improvement action. With respect to quercetin, a tight junction barrier function improvement action has been reported (Non-Patent Literature 1).

As a result, the TEER reduction suppression ratios of gallic acid and quercetin were 86.5% and 75.0%, respectively. It was confirmed that the TEER reduction suppression ratio of gallic acid at 100 μM is about 1.15 times higher than that of quercetin at the same concentration.

### Example 2

In evaluations 1 to 4, gallic acid and the following phenolic compounds were used as samples.

Evaluation 1: catechin (CA) (Wako Pure Chemical Industries, Ltd.)

Evaluation 2: epicatechin (EC) (Wako Pure Chemical Industries, Ltd.)

Evaluation 3: gallocatechin (GC) (Wako Pure Chemical Industries, Ltd.)

Evaluation 4: epigallocatechin (EGC) (Wako Pure Chemical Industries, Ltd.)

The phenolic compounds used in Example 2 are flavanols.

In the evaluations 1 to 4, a TEER reduction suppression ratio (%) in Caco-2 cells was evaluated for each of the samples shown in (i) to (iii) below according to the above evaluation method.

(i) phenolic compound (CA, EC, GC, or EGC) (1 μM)

(ii) gallic acid (GA) (1 μM)

(iii) combined use of phenolic compound (1 μM) and gallic acid (1 μM) (phenolic compound+GA)

The sample was added into the test solution so that a concentration in the test solution was set to the above concentration. The evaluation results are shown in Table 1.

TABLE 1

| | | | Concen- | Evaluatin results | |
|---|---|---|---|---|---|
| Evaluation | | | tration | Relative | Synergistic |
| No. | | Compound | [μM] | value | effect |
| 1 | (i) | Catechin (CA) | 1 | 1.0 | 2.0 |
| | (ii) | Gallic acid (GA) | 1 | 0.4 | |
| | — | CA + GA | 1 each | 1.4 | |
| | | (theoretical value) | | | |
| | (iii) | CA + GA | 1 each | 2.8 | |
| | | (actual measurement value) | | | |
| 2 | (i) | Epicatechin (EC) | 1 | 1.0 | 1.7 |
| | (ii) | Gallic acid (GA) | 1 | 0.5 | |
| | — | EC + GA | 1 each | 1.5 | |
| | | (theoretical value) | | | |
| | (iii) | EC + GA | 1 each | 2.5 | |
| | | (actual measurement value) | | | |
| 3 | (i) | Galloatechin (GC) | 1 | 1.0 | 2.5 |
| | (ii) | Gallic acid (GA) | 1 | 0.3 | |
| | — | GC + GA | 1 each | 1.3 | |
| | | (theoretical value) | | | |
| | (iii) | GC + GA | 1 each | 3.2 | |
| | | (actual measurement value) | | | |
| 4 | (i) | Epigallocatechin (EGC) | 1 | 1.0 | 1.5 |
| | (ii) | Gallic acid (GA) | 1 | 0.2 | |
| | — | EGC + GA | 1 each | 1.2 | |
| | | (theoretical value) | | | |
| | (iii) | EGC + GA | 1 each | 1.8 | |
| | | (actual measurement value) | | | |

Relative values shown in Table 1 are relative values of the TEER reduction suppression ratios (%) of (i) to (iii) when the TEER reduction suppression ratio (%) of (i) the phenolic compound used in each of the evaluations 1 to 4 is set to 1.0.

With respect to (iii) the combined use of the phenolic compound and gallic acid, Table 1 shows a relative value of an actual measurement value (phenolic compound 4-gallic acid (actual measurement value)) and a relative value of a theoretical value (phenolic compound+gallic acid (theoretical value)). The relative value of the actual measurement value ((TEER reduction suppression ratio (actual measurement value) of (iii))/(TEER reduction suppression ratio of (i) phenolic compound)) was calculated from the TEER reduction suppression ratio obtained in actual evaluation (actual measurement value: M.V.). The relative value of the theoretical value (phenolic compound+gallic acid (theoretical value)) was determined as follows: the sum of the TEER reduction suppression ratio of (i) the phenolic compound and the TEER reduction suppression ratio of (ii) gallic acid ((TEER reduction suppression ratio of (i))+(TEER reduction suppression ratio of (ii.))) was taken as a “theoretical value” (T. V.) of the TEER reduction suppression ratio in the case of the combined use of the phenolic compound and gallic acid; and the theoretical value of the TEER reduction suppression ratio was divided by the TEER reduction suppression ratio of (1) the phenolic compound.

The synergistic effect in Table 1 was calculated according to the following formula from the relative value of the actual measurement value of (iii), and the relative value of the theoretical value.

Synergistic effect=(Relative value of actual measurement value)/(Relative value of theoretical value)

With respect to the results shown in Table 1, the evaluation 1 using catechin as the phenolic compound is described as an example.

The relative value is a relative value of a TEER reduction suppression ratio when the TEER reduction suppression ratio of (i) catechin (CA) is set to 1.0. The relative value of the TEER reduction suppression ratio of (ii) gallic acid (GA) was 0.4. The relative value of the theoretical value of the TEER reduction suppression ratio in the case of the combined use (CA+GA) of catechin and gallic acid ((CA+GA) (theoretical value)) was 1.4. The relative value of the theoretical value was determined by dividing the sum of the TEER reduction suppression ratio of (i) CA and the TEER reduction suppression ratio of (ii) GA ((i)+(ii)) by the TEER reduction suppression ratio of (i) CA. The relative value of an actual measurement value ((CA+GA) (actual measurement value)) was calculated from the TEER reduction suppression ratio of (iii) (CA+GA) obtained in actual evaluation, and was 2.8. The synergistic effect was determined by dividing the relative value (2.8) of the above actual measurement value by the relative value (1.4) of the theoretical value.

From Table 1, it was found that the intestinal barrier function improvement effect (actual measurement value) obtained by (iii) the combined use of gallic acid and the above phenolic compound is higher than a calculational effect (theoretical value) obtained by adding the intestinal barrier function improvement effect in the case of (i) the phenolic compound alone to the intestinal barrier function improvement effect in the case of (ii) gallic acid alone.

### Example 3

In the above evaluation method using Caco-2 cells, intestinal barrier function improvement effects were evaluated using gallic acid and phenolic compounds shown in Tables 2 and 3 as samples (evaluations 5 to 43).

As with Example 2, the TEER reduction suppression ratio (%) was evaluated with the following (1) to (iii) as samples in each evaluation system.

(i) phenolic compounds (compounds shown in Table 2: 10 μM; compounds shown in Table 3: 1 μM)

(ii) gallic acid (10 μM in evaluation system of compounds shown in Table 2; 1 μM in evaluation system of compounds shown in Table 3)

(iii) combined use of phenolic compound and gallic acid (each 10 μM in evaluation system of compounds shown in Table 2; each 1 μM in evaluation system of compounds shown in Table 3)

The sample was added into the test solution so that a concentration in the test solution was set to the above concentration.

With respect to (iii) the combined use of the phenolic compound and gallic acid, the TEER reduction suppression ratio (actual measurement value: M.V.) obtained in the above evaluation was shown as “4 gallic acid (actual measurement value)” in Tables 2 and 3.

As with Example 2, the sum of the TEER reduction suppression ratio of (i) the phenolic compound and the TEER reduction suppression ratio of (ii) gallic acid ((TEER reduction suppression ratio of (i))+(TEER reduction suppression ratio of (ii))) was taken as the “theoretical value” of the TEER reduction suppression ratio in the case of the combined use of the phenolic compound and gallic acid (the theoretical value of the TEER reduction suppression ratio in the case of the combined use of the phenolic compound and gallic acid). The theoretical value of the TEER reduction suppression ratio was shown as “+gallic acid (theoretical value)” in Tables 2 and 3. The synergistic effect shown in Tables 2 and 3 was calculated according to the following formula from “+gallic acid (actual measurement value)” (M. V.) and “+gallic acid (theoretical value)” (T. V.).

Synergistic effect=(+Gallic acid (actual measurement value) (M. V.))/(+Gallic acid (theoretical value) (T. V.))

The results are shown in Tables 2 and 3. The “phenolic compound” is a phenolic compound used in each evaluation system in Tables 2 and 3.

Table 2 shows results in which the concentrations of the phenolic compound and gallic acid in the test solution are 10 μM. Table 3 shows results in which the concentrations of the phenolic compound and gallic acid in the test solution are 1 μM. When gallic acid was used alone (the above (ii)), gallic acid suppressed the reduction of TEER due to inflammatory cytokine in Caco-2 cells at any concentration of 1 μM and 10 μM.

TABLE 2

| | | | TEER reduction | | |
|---|---|---|---|---|---|
| | | | suppression ratio (%) | | |
| | | | | +gallic acid | |
| | | | +gallic acid | (actual | Synergistic |
| Evaluation | | | (theoretical | measurement | effect |
| No. | Phenolic compount | Classification | value) (T.V.) | value) (M.V.) | (M.V./T/V/) |
| 5 | Procyanidin B1 | Flavan-3-ol | 31 | 74 | 2.4 |
| 6 | Procyanidin B2 | polymer | 51 | 76 | 1.5 |
| 7 | Procyanidin B3 | | 53 | 77 | 1.9 |
| 8 | 2-Coumaric acid | Coumaric acids | 15 | 46 | 3.0 |
| 9 | 3-Coumaric acid | | 41 | 48 | 1.2 |
| 10 | Daidzein | Isoflavones | 28 | 50 | 1.8 |
| 11 | Genistein | | 37 | 58 | 1.6 |
| 12 | Apigenin | Flavones | 12 | 51 | 4.1 |
| 13 | Luteolin | | 36 | 58 | 1.6 |
| 14 | Naringenin | Flavanones | 31 | 61 | 2.0 |
| 15 | Naringenin chalcone | Chalcones | 31 | 51 | 1.7 |
| 16 | Kaempferol | Flavonols | 17 | 43 | 2.5 |
| 17 | Rutin | | 16 | 45 | 2.9 |
| 18 | Quercetin-3-O-glucopyranoside | | 32 | 47 | 1.5 |
| 19 | Quercetin | | 36 | 61 | 1.7 |
| 20 | Myricetin | | 62 | 75 | 1.2 |
| 21 | Theaflavin | Flavanols | 60 | 98 | 1.6 |
| 22 | Catechin gallate | | 43 | 101 | 2.3 |
| 23 | Epicatechin gallate | | 48 | 110 | 2.3 |
| 24 | Epigallocatechin gallate | | 76 | 114 | 1.5 |
| 25 | Taxifolin | Flavanonols | 29 | 59 | 2.0 |
| 26 | Piceatannol | Stilbenoids | 47 | 53 | 1.1 |
| 27 | trans-Piceid | | 45 | 65 | 1.5 |
| 28 | Petunidin chloride | Anthocyanidins | 38 | 70 | 1.9 |

TABLE 3

| | | | TEER reduction | | |
|---|---|---|---|---|---|
| | | | suppression ratio (%) | | |
| | | | | +gallic acid | |
| | | | +gallic acid | (actual | Synergistic |
| Evaluation | | | (theoretical | measurement | effect |
| No. | Phenolic compound | Classification | value) (T.V.) | value) (M.V.) | (M.V./T.V.) |
| 29 | Corilagin | Hydrolyzable | 23 | 76 | 3.4 |
| 30 | Eugeniflorin D2 | tannin | 80 | 102 | 1.3 |
| 31 | Stenophyllanin A | (allagitannin) | 56 | 95 | 1.7 |
| 32 | Stenophyllanin B | | 66 | 82 | 1.3 |
| 33 | Casuarinin | | 57 | 81 | 1.4 |
| 34 | Geraniin | | 50 | 119 | 2.4 |
| 35 | Tellimagrandin I | | 59 | 92 | 1.6 |
| 36 | Pedunculagin | | 54 | 90 | 1.7 |
| 37 | Praecoxin A | | 74 | 104 | 1.4 |
| 38 | 1,2,4,6-tetra-O-galloyl-β-D- | Hydrolyzable | 41 | 56 | 1.4 |
| | glucose | tannin | | | |
| 39 | 1,2,3,6-tetra-O-galloyl-β-D- | (gallotannin) | 52 | 81 | 1.5 |
| | glucose | | | | |
| 40 | 2,3,4,6-tetra-O-galloyl-β-D- | | 45 | 90 | 2.0 |
| | glucose | | | | |
| 41 | 1,2,3,4,6-penta-O-galloyl-β-D- | | 65 | 91 | 1.4 |
| | glucose | | | | |
| 42 | 1,4,6-tri-O-galloyl-β-D- | | 24 | 40 | 1.7 |
| | glucose | | | | |
| 43 | β-glucogallin | | 20 | 48 | 2.4 |

Also with respect to the phenolic compounds shown in Tables 2 and 3, the intestinal barrier function improvement effect (actual measurement value) obtained by (iii) the combined use with gallic acid was higher than a calculational effect (theoretical value) obtained by adding the intestinal barrier function improvement effect in the case of (i) the phenolic compound alone to the intestinal barrier function improvement effect in the case of (ii) gallic acid alone.

As the phenolic compounds described in Table 2, the following manufacturers' reagents were used.

procyanidin B1: AdooQ BioScience Corporation

procyanidin B2: Toronto Research Chemicals Inc.

procyanidin B3, quercetin, quercetin-3-O-glucopyranoside, naringenin: Funakoshi Co., Ltd.

2-coumaric acid, 3-coumaric acid: ChromaDex Corporation

genistein: Sigma-Aldrich Co. LLC.

apigenin, luteolin, kaempferol, myricetin, taxifolin: EXTRASYNTHESE CORPORATION

rutin, trans-piceid: Nacalai Tesque, Inc.

daidzein, petunidin chloride: Cayman Chemical Company

catechin gallate, epicatechin gallate, epigallocatechin gallate: Wako Pure Chemical Industries, Ltd.

theaflavin: Cosmo Bio Co., Ltd.

piceatannol: Tokyo Chemical Industry Co., Ltd.

naringenin chalcone (4,2′,4′,6′-tetra-hydroxy chalcone): Carbosynth Limited

Among the hydrolyzable tannins described in Table 3, corilagin manufactured by Sigma-Aldrich Co. LLC. was used, and β-glucogallin manufactured by Carbosynth (CAB) Limited was used. As the hydrolyzable tannins other than these, hydrolyzable tannins purified from plants (all: purity of 90% or more) were used. For example, tellimagrandin I and gallotannin were purified from eucalyptus cypellocarpa leaves. Geraniin was purified from geranium thunbergii leaves. Other hydrolyzable tannins purified from myrtaceae plants such as kunzea ambigua leaves and melaleuca alternifolia were used. The hydrolyzable tannins were purified from the plants by the method described in p. 76 to 95 of Literature (KASAJ1MA Naoki, “Ingredient Research of Kunzea Ambigua and Eucalyptus Cypellocarpa” (publication: September, 2005, doctoral dissertation, Okayama University)). The structures of the hydrolyzable tannins described in Table 3 are shown in Tables 4 and 5.

TABLE 4

| Eval- | | |
|---|---|---|
| uation | Phenolic | |
| No. | compound | Structural formula |
| 29 | Corilagin | 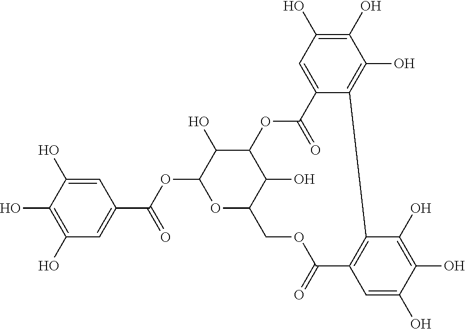 [SMILES: O=C(OC1OC2COC(=O)c3cc(O)c(O)c(O)c3-c3c(cc(O)c(O)c3O)C(=O)OC(C2O)C1O)c1cc(O)c(O)c(O)c1] |
| 30 | Eugeniflorin D2 | 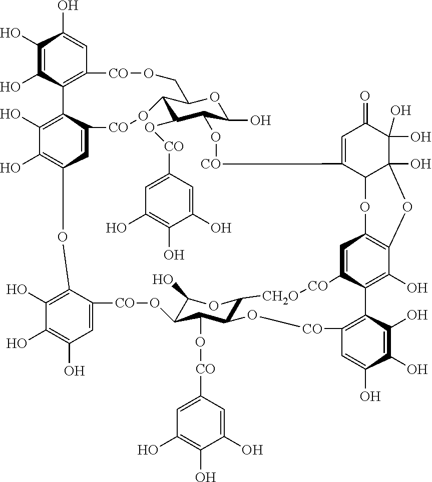 |
| 31 | Stenophyllanin A | 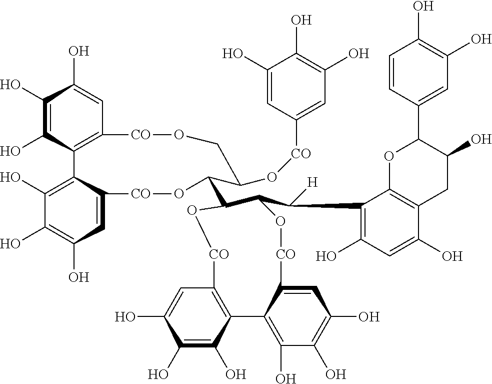 |
| 32 | Stenophyllanin B | 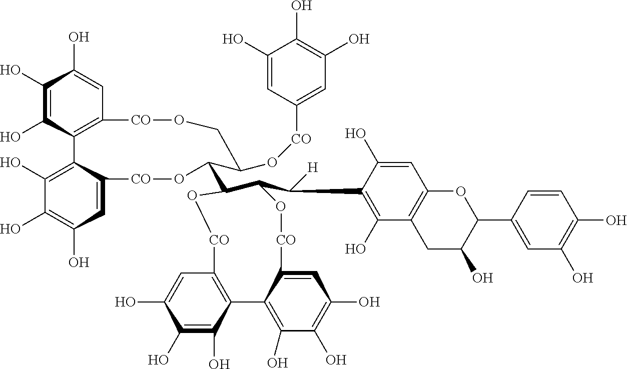 |
| 33 | Casuerinin | 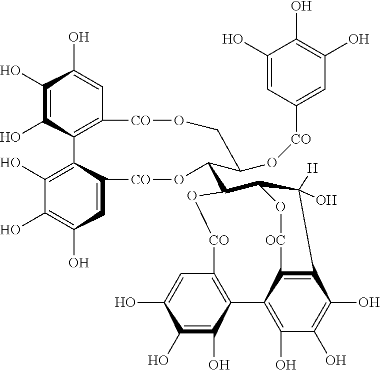 |
| 34 | Geraniin | 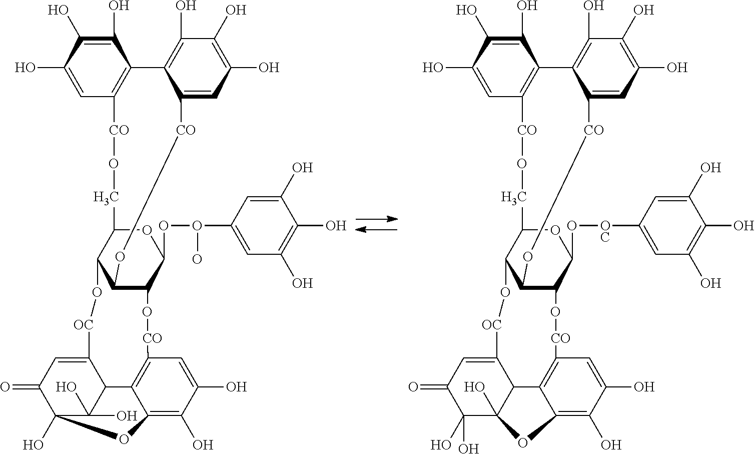 |
| 35 | Tellimagrandin I | 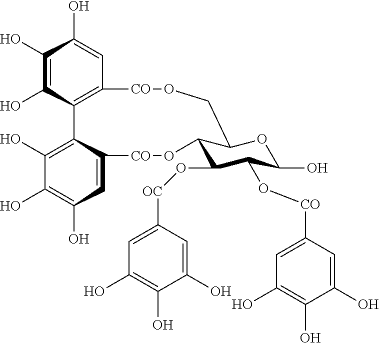 |
| 36 | Pedunculagin | 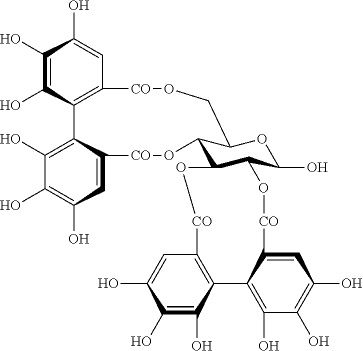 |
| 37 | Praecoxin A | 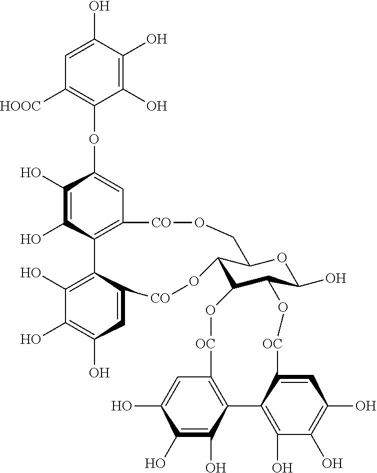 |

TABLE 5

| Evaluation | Phenolic | |
|---|---|---|
| No. | compound | Structural formula |
| 38 | 1,2,4,6-tetra-o- galloyl-β-D-glucose | 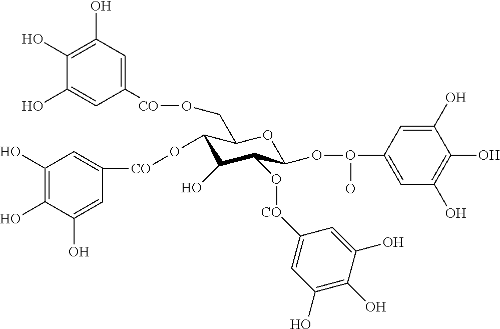 |
| 39 | 1,2,3,6-tetra-o- galloyl-β-D-glucose | 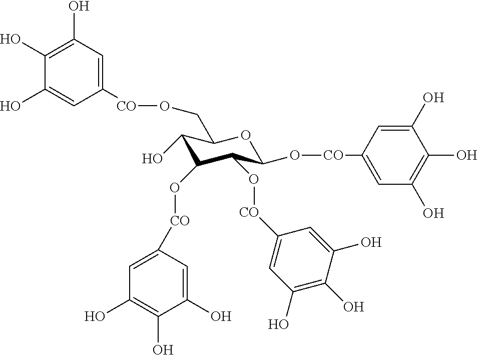 |
| 40 | 2,3,4,6-tetra-o- galloyl-β-D- glucose | 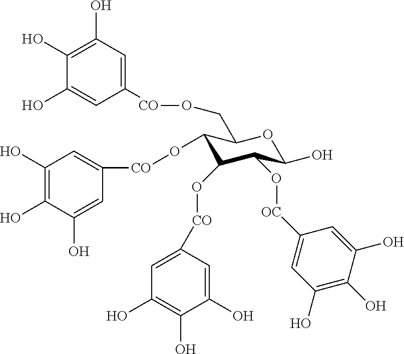 |
| 41 | 1,2,3,4,6-penta-o- galloyl-β-D-glucose | 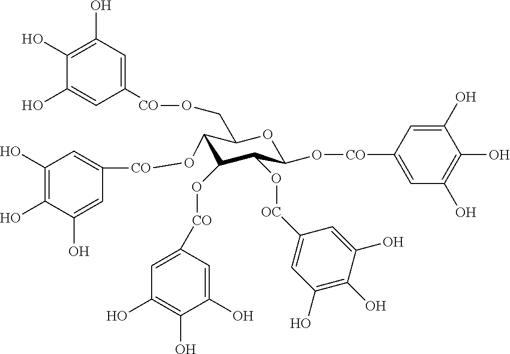 |
| 42 | 1,4,6-tri-o-galloyl-β- D-glucose | 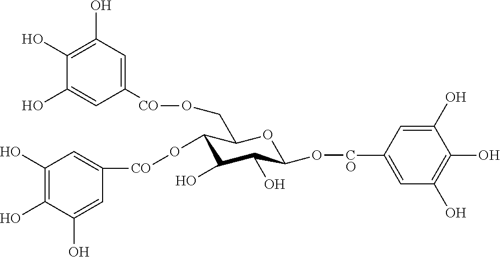 |
| 43 | β-glucogallin | 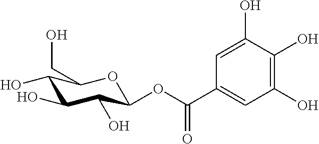 |

### INDUSTRIAL APPLICABILITY

A composition for improving intestinal barrier function, and the like, according to the present invention are useful in the food or beverage field, the medicine field and the like.

1. A composition for improving intestinal barrier function, comprising:
gallic acid as an active ingredient.

2. The composition for improving intestinal barrier function according to claim 1, further comprising a phenolic compound.

3. The composition for improving intestinal barrier function according to claim 2,
wherein the phenolic compound is a polyphenol and/or a coumaric acid.

4. The composition for improving intestinal barrier function according to claim 3,
wherein the polyphenol is at least one compound selected from the group consisting of a flavan-3-ol polymer, flavanols, flavonols, flavanones, flavones, isoflavones, anthocyanidins, flavanonols, stilbenoids, chalcones, and a hydrolyzable tannin.

5. The composition for improving intestinal barrier function according to claim 2,
wherein the phenolic compound is at least one compound selected from the group consisting of procyanidin B1, procyanidin B2, procyanidin B3, catechin, epicatechin, gallocatechin, epigallocatechin, catechin gallate, epicatechin gallate, epigallocatechin gallate, theaflavin, taxifolin, daidzein, genistein, apigenin, luteolin, naringenin, naringenin chalcone, kaempferol, rutin, quercetin-3-O-glucopyranoside, quercetin, myricetin, piceatannol, petunidin, trans-piceid, corilagin, stenophyllanin A, stenophyllanin B, casuarinin, geraniin, tellimagrandin I, pedunculagin, praecoxin A, eugeniflorin D2,1,4,6-tri-O-galloyl-β-D-glucose, 1,2,3,6-tetra-β-galloyl-β-D-glucose, 2,3,4,6-tetra-O-galloyl-β-D-glucose, 1,2,4,6-tetra-O-galloyl-β-D-glucose, 1,2,3,4,6-penta-O-galloyl-β-D-glucose, β-glucogallin, 2-coumaric acid, and 3-coumaric acid.

6. The composition for improving intestinal barrier function according to claim 1,
wherein the composition for improving intestinal barrier function is an oral composition.

7. The composition for improving intestinal barrier function according to claim 6,
wherein the oral composition is a food or beverage, a pharmaceutical product, or a quasi-pharmaceutical product.

8. The composition for improving intestinal barrier function according to claim 1,
wherein the composition is used for intestinal regulation.

9. The composition for improving intestinal barrier function according to claim 1,
wherein the composition is labeled as having an intestinal regulation action.

10. An agent for enhancing an intestinal barrier function improvement action of gallic acid, the agent comprising:
a phenolic compound as an active ingredient.

11. A method for improving intestinal barrier function, the method comprising:
administering gallic acid to a subject.

12. Use of gallic acid for improving intestinal barrier function.

13. A method for enhancing an intestinal barrier function improvement action of gallic acid, the method comprising:
administering a combination of gallic acid with a phenolic compound to a subject.

14. Use of a phenolic compound for enhancing an intestinal barrier function improvement action of gallic acid.
